(19) United States
(12) Patent Application Publication
(10) Pub. No.: US 2008/0175877 A1
(43) Pub. Date: Jul. 24, 2008

(54) ADHESIVES WITH ANTIPERSPIRANT AND/OR ANTI-INFLAMMATORY ADDITIVES

(75) Inventor: W. Kenneth Ward, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Wilsonville, OR (US)

(21) Appl. No.: 12/015,979

(22) Filed: Jan. 17, 2008

Related U.S. Application Data
(60) Provisional application No. 60/886,003, filed on Jan. 22, 2007.

(51) Int. Cl. A61K 8/26; A61K 8/28; A61K 8/02; A61Q 15/00

(52) U.S. Cl. 424/401; 424/68; 424/65; 424/66; 424/682

(57) ABSTRACT

Embodiments of the present invention provide adhesives comprising additives, such as anti-inflammatory and antiperspirant additives, to address various problems a user may encounter when contacting the adhesive or wearing a bandage or other device/substrate using such an adhesive.

### CROSS REFERENCE TO RELATED APPLICATIONS

The present application claims priority to U.S. Provisional Patent Application No. 60/886,003, filed Jan. 22, 2007, entitled “Adhesives and Bandages Containing Antiperspirant and/or Anti-inflammatory Additives,” the entire disclosure of which is hereby incorporated by reference in its entirety.

### TECHNICAL FIELD

Embodiments of the present invention relate to the field of adhesives, and, more specifically, to adhesives containing additives, such as antiperspirant and/or anti-inflammatory additives.

### BACKGROUND

Adhesives are used in a variety of medical applications. In general, adhesives serve a role to adhere components together and/or to adhere one or more components to a patient. However, adhesive properties may suffer for a variety of reasons, such as perspiration of the patient, or may cause various side effects, such as skin irritation.

Perspiration may be a major problem for patients who are wearing a device that is taped or adhered to the skin. Water may build up beneath the dressing/bandage and reduce adherence or cause the bandage to lift off the skin. In the case of a biosensor that is kept in place or anchored by a bandage, this may create a major problem because dislodgement of the bandage may lead to dislodgement (full or partial) of the sensor itself. The problem is greatest in those individuals with hyperhidrosis (abnormal amount of sweating).

In addition, bandages/adhesives may cause skin irritation over time as the skin responds or reacts to the presence of the adhesive/bandage, specifically due to friction/rubbing by the bandage, chemical components in the adhesive, etc. Irritation may be expected in approximately 5-15% of users of a variety of bandages/adhesives.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 illustrates an adhesive with an additive dispersed therein in accordance with various embodiments of the present invention;

FIG. 2 illustrates a multi-layered adhesive in which a skin-contact layer is provided with one or more additives in accordance with various embodiments of the present invention;

FIG. 3 illustrates an adhesive with an additive dispersed therein and applied to a substrate in accordance with various embodiments of the present invention; and

FIGS. 4A, 4B, and 4C illustrate various patterns of adhesive on a substrate in accordance with various embodiments of the present invention.

### DETAILED DESCRIPTION OF EMBODIMENTS OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention provide adhesives containing additives to address various problems a user may encounter when contacting the adhesive or when wearing a bandage using such an adhesive. The term “bandage” is used broadly to refer to any suitable type of bandage or patch, regardless of material, whether or not the bandage is sold with an adhesive or adhesive is applied by a medical professional, patient/user, etc.

In embodiments, adhesives may be used to adhere bandages to skin, or may be used to adhere other physical components, such as a biosensor, an on-skin electronics unit, etc. to skin. In an embodiment, adhesive may be protected post-manufacture and before use by covering the adhesive with a suitable release liner that may be easily removed when ready for use.

In an embodiment, an additive may be dispersed in the adhesive, or an additive may be present in (such as dispersed in) one or more layers of a multi-layered adhesive, for example in the skin-contact layer.

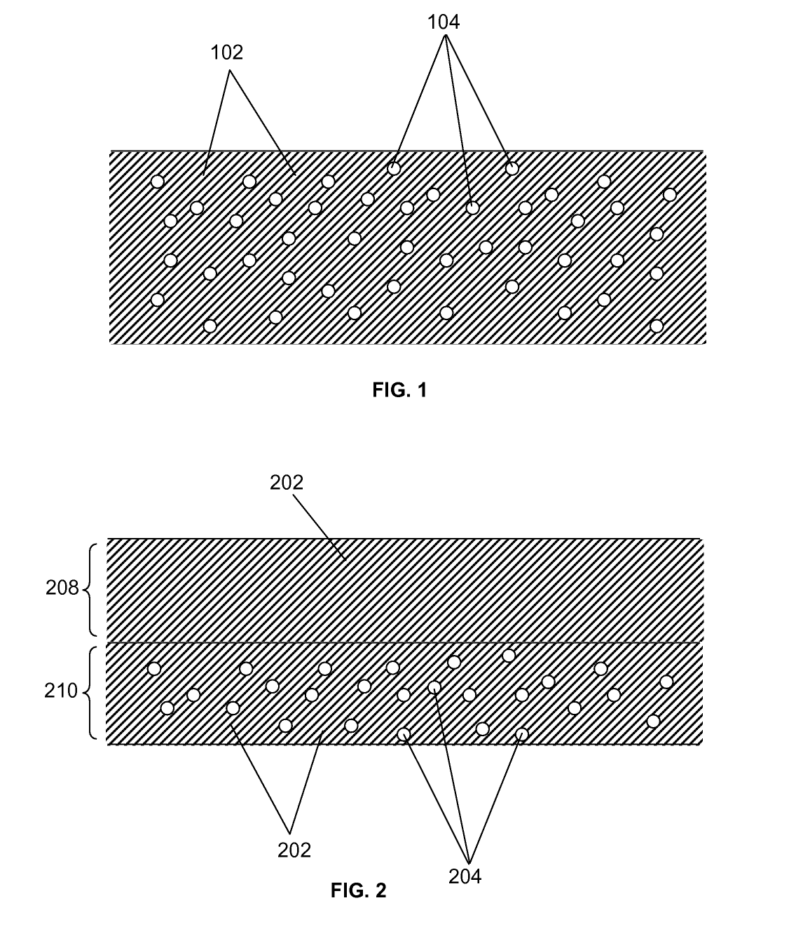

FIG. 1 illustrates an adhesive 102 with an additive 104 dispersed therein in accordance with various embodiments of the present invention. The particles that comprise additive 104 may be dispersed in adhesive 102 uniformly or nonuniformly. In an embodiment, the particles that comprise additive 104 may include one or more than one type of additive, such as antiperspirant and anti-inflammatory additives.

FIG. 2 illustrates a multi-layered adhesive in which a skin-contact layer 210 is provided with one or more additives in accordance with an embodiment of the present invention. Skin-contact layer 210 includes an adhesive 202 with at least one additive 204 dispersed therein. In embodiments, providing additive 204 in layer 210 places additive 204 in relatively close proximity to skin when in use, as opposed to being located in layer 208. In embodiments, the particles that comprise additive 204 may be dispersed in adhesive 202 uniformly or nonuniformly. In an embodiment, additional layers of adhesive 202, such as layer 208, may be provided to increase the adherence. As shown in FIG. 2, layer 208 may be provided without any additives, or, alternatively, one or more additives may be provided in layer 208 as well. In embodiments, each layer may comprise the same adhesive, or more than one adhesive may be used for the layers. In addition, in an embodiment, while FIG. 2 shows two adhesive layers, more than two adhesive layers may be used.

In an embodiment, an additive may be embedded in a bandage material matrix in a variety of manners, or may be present on or layered on the bandage material in an adhesive or in another suitable manner.

FIG. 3 illustrates an adhesive 302 with additive 304 on substrate 306, such as a bandage, on-skin device, monitoring device, etc. Additive 304 may be dispersed in adhesive 302 in accordance with various embodiments of the present invention. In embodiments, adhesive 302 may be applied to substrate 306 during manufacture or may be separately applied by a user, medical professional, etc.

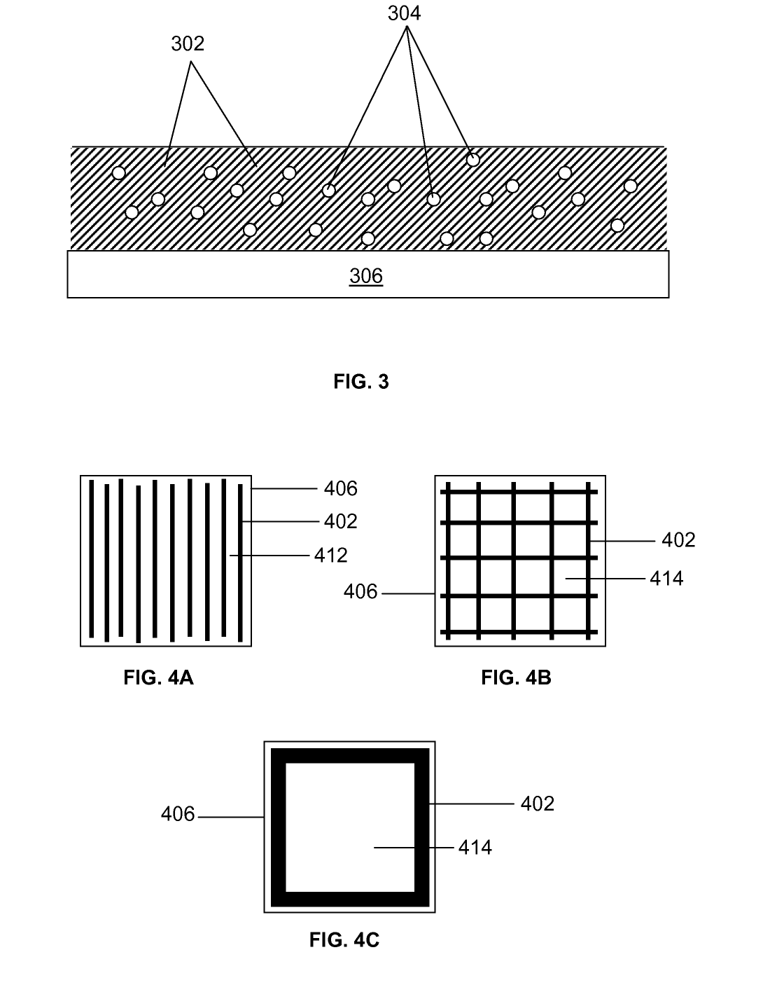

FIGS. 4A, 4B, and 4C illustrate various patterns of adhesive 402 on a substrate 406, such as a bandage, on-skin device, monitoring device, etc. Using certain patterns may reduce the amount of adhesive used, and may provide additional benefits, such as reducing irritation, allowing the skin to “breathe” by reducing the number of pores that are occluded, etc. FIG. 4A shows a channel pattern in which adhesive 402 is applied in rows forming channels 412 between the rows. For the purposes of describing embodiments of the present invention, the term “channel” refers to a pathway defined between ridges or rows of adhesive through which liquid or vapor may travel. A channel may be bounded by adhesive ridges/rows on two or three sides. In an embodiment, the channels may permit moisture to escape along the channels and out from under substrate 406. FIG. 4B shows a crossing pattern which may increase the adherence of substrate 406 to a target surface (such as skin) while leaving various voids 414 still leaving some of the target surface uncovered. For the purposes of describing embodiments of the present invention, the term “void” refers to an area absent adhesive but completely bounded by adhesive. FIG. 4C shows a simple square pattern of adhesive 402. In such an embodiment, a void may be left in the center of the adhesive/bandage, as the outer border of adhesive 402 may be sufficient to ensure proper adherence to a target surface. In embodiments, when one or more patterns of adhesive are used on a bandage or other substrate, the substrate may also have pores, perforations, voids, etc. that differ from or match the pattern(s) of the adhesive to further permit the movement of liquid or vapor away from the skin.

In other embodiments, other patterns of adhesive application, including non-uniform or random patterns, may be utilized.

In embodiments, suitable adhesives include medical adhesives, acrylics, cyanoacrylates, methacrylates, fibrin glue, etc.

In embodiments, suitable additives include antiperspirants and anti-inflammatory compounds. In an embodiment, an additive may be provided in an amount of approximately 1-10% by weight of the adhesive, such as 1-5% by weight of the adhesive.

For the purposes of describing embodiments of the invention, the term “substantial activity” refers to the extent of activity of the additive during or after use of the adhesive/additive, as compared to before use, and, in embodiments is in excess of 80% or in excess of 90%.

In embodiments, a method and apparatus for reducing perspiration may be provided. In such embodiments, an antiperspirant additive, such as an aluminum salt, may be dispersed in the adhesive or incorporated into at least the skin-contact layer of a multi-layered adhesive. In an embodiment, when using an aluminum salt, aluminum ions may be released from the adhesive and may be taken-up by sweat glands in the skin, thus reducing perspiration. In an embodiment, the resultant reduction in moisture helps to prevent dislodgement or movement of an associated bandage or device on the skin surface.

In an embodiment, various aluminum salts may be used as antiperspirants including aluminum chloride, aluminum zirconium tricholorohydrex glycine, aluminum chlorohydrate, aluminum hydroxybromide, etc. In another embodiment, zirconium chlorohydrate may be used as an antiperspirant.

In an embodiment, an aluminum salt or other antiperspirant may be provided in a slow-release formulation to provide controlled release of the salt over a period of time, such as 3-7 days or more. In an embodiment, an antiperspirant may maintain substantial activity while in use for a period of 3-7 days or more.

In an embodiment, moisture (whether in the form of sweat or other forms of moisture present) concerns may be addressed by introducing a hygroscopic compound into a bandage or including such a compound with or around an adhesive. A hygroscopic compound may be used to absorb sweat, rain water, shower water, etc.

In addition to concerns related to dislodgement of a bandage/device, bandages or devices adhered to skin with adhesive may cause skin irritation. In an embodiment, to address skin irritation, a bandage or adhesive may be provided with an anti-inflammatory additive, such as a corticosteroid and/or an antihistamine.

In an embodiment, a small amount of a corticosteroid, such as hydrocortisone or other anti-inflammatory compound may be dispersed in an adhesive. In an embodiment, an antihistamine, such as diphenhydramine, may be dispersed in an adhesive.

In an embodiment, relatively weak corticosteroids may be used to allow for more prolonged use. In an embodiment, a more active corticosteroid, such as a fluorinated corticosteroid, for example Lidex™, may be used; however, with prolonged use, it may cause skin atrophy over time.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

1. An adhesive, comprising:
at least one compound for adhering a substrate to skin; and
at least one antiperspirant additive combined with said compound, wherein said at least one additive is dispersed in said adhesive.

2. The adhesive of claim 1, wherein said at least one antiperspirant additive is dispersed uniformly in said adhesive.

3. The adhesive of claim 1, wherein said at least one antiperspirant additive is dispersed non-uniformly in said adhesive.

4. The adhesive of claim 1, wherein said at least one antiperspirant additive is present in an amount of approximately 1-10% by weight of the adhesive.

5. The adhesive of claim 1, wherein said at least one antiperspirant additive is present in an amount of approximately 1-5% by weight of the adhesive.

6. (canceled)

7. The adhesive of claim 1, wherein said at least one antiperspirant additive comprises an aluminum salt.

8. The adhesive of claim 7, wherein said aluminum salt comprises aluminum chloride, aluminum zirconium tricholorohydrex glycine, aluminum chlorohydrate, or aluminum hydroxybromide.

9. The adhesive of claim 1, wherein said adhesive further comprises an anti-inflammatory compound.

10. The adhesive of claim 9, wherein said anti-inflammatory compound comprises a corticosteroid or an antihistamine.

11. The adhesive of claim 1, wherein said at least one antiperspirant additive is provided in a slow release formulation.

12. The adhesive of claim 1, wherein said at least one antiperspirant additive maintains substantial activity for a period of 3 to 7 days or more.

13. (canceled)

14. A multi-layered adhesive, comprising:
a skin-contact layer of a first adhesive, said first adhesive comprising at least one compound for adhering a substrate to skin, and at least one antiperspirant additive combined with said compound, wherein said at least one antiperspirant additive is dispersed in said first adhesive; and
at least one additional layer of adhesive in contact with said skin-contact layer.

15. The multi-layered adhesive of claim 14, wherein said skin-contact layer of a first adhesive and said at least one additional layer of adhesive comprise the same adhesive.

16. The multi-layered adhesive of claim 14, wherein said skin-contact layer of a first adhesive comprises an adhesive that is different from at least one adhesive in said at least one additional layer of adhesive.

17. The multi-layered adhesive of claim 14, wherein said at least one additional layer of adhesive does not comprise any additives.

18. The multi-layered adhesive of claim 14, wherein said at least one additional layer of adhesive comprises at least one additive that is different from said at least one antiperspirant additive in said first adhesive.

19. An adhesive body, comprising:
a substrate adapted to be attached and/or adhered to skin directly or indirectly; and
an adhesive applied to said substrate, said adhesive comprising at least one compound for adhering the substrate to the skin, and at least one antiperspirant additive combined with said compound, wherein said at least one antiperspirant additive is dispersed in said adhesive.

20. The adhesive body of claim 19, wherein said substrate comprises a bandage.

21. The adhesive body of claim 19, wherein said substrate comprises an on-skin electronic device.

22. The adhesive body of claim 19, wherein said adhesive is applied to said substrate in a pattern forming at least one channel.

23. The adhesive body of claim 19, wherein said adhesive is applied to said substrate in a pattern forming at least one void.

24. The adhesive body of claim 19, wherein said at least one antiperspirant additive comprises an aluminum salt.

25. The adhesive body of claim 19, wherein said adhesive further comprises at least one anti-inflammatory compound.

26. The adhesive body of claim 25, wherein said anti-inflammatory compound comprises a corticosteroid or an antihistamine.
